(19) United States
(12) Patent Application Publication
(10) Pub. No.: US 2024/0226607 A1
(43) Pub. Date: Jul. 11, 2024

(54) SKIN CARE DEVICE

(71) Applicant: LG ELECTRONICS INC., Seoul (KR)

(72) Inventors: Dugsang KIM, Seoul (KR); Hyoungjun KIM, Seoul (KR); Woohyun KIM, Seoul (KR); Kyoungbok YUN, Seoul (KR); Yonggeun JIN, Seoul (KR)

(73) Assignee: LG ELECTRONICS INC., Seoul (KR)

(21) Appl. No.: 18/528,141

(22) Filed: Dec. 4, 2023

(30) Foreign Application Priority Data
Jan. 9, 2023 (KR) 10-2023-0002661
Jan. 9, 2023 (KR) 10-2023-0002731
Mar. 31, 2023 (KR) 10-2023-0043160

(51) Int. Cl. A61N 7/00

(52) U.S. Cl. CPC A61N 7/00; A61N 2007/0034

(57) ABSTRACT

A cradle holds a skin care device, the skin care device including a header for accommodating a medium fluid and having an emitting hole positioned on an emitting surface. The cradle can include a head seating portion, the header is inserted into the head seating portion, and a damper ring disposed around a side perimeter of the head seating portion and adhering closely to a perimeter of the header. The head seating portion can include a seating portion bottom surface facing the emitting hole of the header, and a seating portion side surface connected to a perimeter of the seating portion bottom surface.

### CROSS-REFERENCE TO RELATED APPLICATIONS

This application claims priority to Korean Patent Application No. 10-2023-0002661 filed in the Republic of Korea on Jan. 9, 2023, Korean Patent Application No. 10-2023-0002731 filed in the Republic of Korea on Jan. 9, 2023 and Korean Patent Application No. 10-2023-0043160 filed in the Republic of Korea on Mar. 31, 2023, all of which are hereby incorporated by reference in their entirety for all purposes as if fully set forth herein.

### BACKGROUND OF THE DISCLOSURE

### Technical Field

The present disclosure relates to a skin care device, and more particularly, to a skin care device that promotes skin collagen regeneration by supplying focused ultrasound energy to the skin.

### Discussion of the Related Art

Modern people perceive their appearance as an important factor in informing and expressing themselves to others. As a result, they invest significant amounts of time and money in appearance management to overcome imperfections or flaws in their appearance.

Skin care aims to achieve blemish-free, clean, and smooth skin, and in particular, there is a significant interest in the case of facial skin care among all body parts. Therefore, people aim to maintain clean and healthy facial skin by receiving massages, applying functional skincare products, and using various cleansing products.

Skin care devices are designed to provide microwaves to penetrate the skin, and their effectiveness can be enhanced when used in conjunction with skincare products. However, it is necessary to cover the skin care device to prevent liquid cosmetics from being introduced into its interior. In addition, since the skin care device comes into contact with the skin, it is important to maintain a clean state.

### SUMMARY OF THE DISCLOSURE

Accordingly, the present disclosure is directed to a skin care device that substantially obviates one or more problems due to limitations and disadvantages of the related art.

An objective of the present disclosure is to provide a semi-permanent skin care device that can be used repeatedly based on a replaceable cartridge structure.

Another objective of the present disclosure is to provide a skin care device that maintains cleanliness for the portions that come into contact with the skin.

A further objective of the present disclosure is to provide a skin care device with a structure of covering and protecting a cartridge such that the lifespan of the cartridge is not shortened.

Additional advantages, objects, and features of the disclosure will be set forth in part in the description which follows and in part will become apparent to those having ordinary skill in the art upon examination of the following or can be learned from practice of the disclosure. The objectives and other advantages of the disclosure can be realized and attained by the structure particularly pointed out in the written description and claims hereof as well as the appended drawings.

To achieve these objects and other advantages and in accordance with the purpose of the disclosure, as embodied and broadly described herein, there is provided a cradle configured to hold a skin care device including a header configured to accommodate a medium fluid and having an emitting hole positioned on an emitting surface. The cradle can include: a head seating portion, wherein the header is inserted into the head seating portion; and a silicon damper ring disposed around a side perimeter of the head seating portion and adhering closely to a perimeter of the header. The head seating portion can include: a seating portion bottom surface facing the emitting hole of the header; and a seating portion side surface connected to a perimeter of the seating portion bottom surface, wherein the silicone damper ring is disposed inside the seating portion side surface. The silicon damper ring can be configured to prevent evaporation of the medium fluid by covering a gap between the head seating portion and the header.

The silicon damper ring can include a first silicon damper ring in contact with a perimeter of the surface on which the emitting hole of the skin care device is formed. The first silicon damper ring can include an upper portion in contact with the skin care device, and the upper portion can be positioned a first distance away from the seating portion bottom surface.

The first silicon damper ring can include: a middle portion extending downward from the upper portion along an inside of the seating portion side surface; and a lower portion interposed at a connecting portion between the seating portion bottom surface and the seating portion side surface.

The cradle can include a sterilization lamp having a first irradiation angle and positioned on the seating portion bottom surface, and a size of an irradiation diameter of the sterilization lamp based on the first irradiation angle at the first distance can be greater than an inner perimeter of the upper portion.

The first silicon damper ring can include a middle portion extending downward from the upper portion and having an inclination angle such that a diameter of the middle portion decreases downward, and the inclination angle of the middle portion can be smaller than the first irradiation angle.

The header can include a cartridge side surface having a slope such that the cartridge side surface narrows towards the emitting surface, and the seating portion side surface can narrow towards the seating portion bottom surface in accordance with the cartridge side surface. The silicon damper ring can include a second silicon damper ring in contact with the cartridge side surface of the header.

The second silicon damper ring can have a curved surface such that a central portion thereof protrudes towards an inside thereof.

The cradle can include: a base portion, wherein the head seating portion is positioned on a first side of the base portion; a lower support portion positioned on a second side of the base portion; and a power supply terminal positioned in the lower support portion and in contact with a charging terminal of the skin care device.

The seating portion bottom surface may not be parallel to the base portion, and a first seating surface on which the power supply terminal of the lower support portion is positioned can be perpendicular to the seating portion bottom surface.

In another aspect of the present disclosure, there is provided a skin care device. The skin care device can include: a main body, wherein an ultrasound cartridge is positioned on one side of the main body; and a cradle including a head seating portion, wherein the ultrasound cartridge of the main body is seated on the head seating portion. The ultrasound cartridge can include: a cartridge body including an emitting hole formed on an emitting surface and an internal cavity connected to the emitting hole; a cover film covering the emitting hole; a piezoelectric driver mounted in the internal cavity and configured to generate high-intensity focused ultrasound; a piezoelectric ceramic configured to emit the high-intensity focused ultrasound toward the emitting hole of the cartridge body; and an ultrasonic medium fluid accommodated in the internal cavity. The cradle can include a silicon damper ring disposed around a perimeter of the head seating portion and adhering closely to a perimeter of the ultrasound cartridge. The head seating portion can include: a seating portion bottom surface facing the emitting hole of the ultrasound cartridge; and a seating portion side surface connected to a perimeter of the seating portion bottom surface, wherein the silicone damper ring is disposed inside the seating portion side surface.

In a further aspect of the present disclosure, there is provided a skin care device can include: a main body having a cartridge seating portion on one side thereof; and an ultrasound cartridge detachable from the cartridge seating portion. The ultrasound cartridge can include: a cartridge body having an internal cavity and an emitting hole formed in an emitting surface; a cover film covering the emitting hole; a piezoelectric driver mounted in the internal cavity and configured to generate high-intensity focused ultrasound; a piezoelectric ceramic configured to emit the high-intensity focused ultrasound toward the emitting hole of the cartridge body; a piezoelectric ceramic for irradiating the high-intensity focused ultrasound toward the emitting hole of the cartridge body; and an ultrasonic medium fluid accommodated in the internal cavity. The medium fluid can be filled up to a position lower than a height of the piezoelectric ceramic when frozen in an arrangement in which the emitting hole faces upward.

The ultrasound cartridge can further include: an elastic waterproof ring fitted around a circumference of the cartridge body and having a protrusion with an outer circumference protruding upward; and a cartridge cover inserted from one side of the cartridge body on which the cover film is positioned. The cartridge cover can include a first step portion inside the cartridge cover, wherein a lower portion of the first step portion is drawn in toward an outside of the cartridge cover. The protrusion of the waterproof ring can be deformed by being pressed on a step surface of the first step portion to fill a space between the cartridge body and the cartridge cover.

The cartridge body can include a second step portion formed on an outer circumference of the cartridge body, wherein a lower portion of the second step portion further protrudes. A cross section of the waterproof ring has an L-shaped lower side surface surrounding upper and side surfaces of the second step portion.

The cover film can cover: the emitting surface of the cartridge body on which the emitting hole is formed; and a side surface positioned around the emitting surface. The first step portion can be positioned below an end of the cover film located on the side surface.

The cartridge seating portion can include: a cartridge hook that protrudes from a first side and is inserted inward when a release button is pressed; and a first elastic fastening protrusion that protrudes from a second side opposite to the first side and is inserted when pressure is applied.

The cartridge seating portion can include a pair of second elastic fastening protrusions that are located between the first side and the second side, are formed on opposing third and fourth sides, protrude symmetrically, and are inserted when pressure is applied.

Each of the first elastic fastening protrusion and the second elastic fastening protrusion can have a curved surface and be made of a polyoxymethylene (POM) material.

The second elastic fastening protrusion can be longer than the first elastic fastening protrusion. The second elastic fastening protrusion can include a pair of springs arranged in parallel along a length direction of the second elastic fastening protrusion.

The first elastic fastening protrusion can be positioned closer to a bottom surface of the cartridge seating portion than the cartridge hook.

The ultrasound cartridge can include a cartridge connection terminal located on an opposite side of the emitting hole. The cartridge seating portion can include a main body connection terminal connected to the cartridge connection terminal on the bottom surface.

The skin care device can include: a button hole formed in the main body; a button bracket positioned inside the button hole and having a switch hole and a hook hole; a button top inserted into the button hole and having a button hook inserted and hooked into the hook hole and a tactile boss inserted into the switch hole; an elastic cover coupled to an inside of the button bracket and covering the switch hole and the hook hole; and a tactile switch positioned inside the elastic cover. The elastic cover can include a first portion interposed between the tactile boss and the tactile switch.

The elastic cover can have a groove in which a second portion at a position corresponding to the button hook is drawn inward relative to the first portion.

The cover film can be made of a polyimide material.

The skin care device can include: a gyroscope sensor provided on the main body and configured to measure an orientation of the main body; and a controller configured to: calculate a surface level of a medium fluid within the cartridge body based on the posture of the main body; and limit driving of the piezoelectric driver when the medium fluid does not exist between the piezoelectric ceramic and the emitting hole of the cartridge body.

The skin care device can include: a gyroscope sensor provided on the main body and configured to measure an orientation of the main body; and a controller configured to: calculate a surface level of a medium fluid within the cartridge body based on the posture of the main body; and provide a user with a voice instruction for changing the posture of the main body.

According to an embodiment of the present disclosure, the structure capable of easily replacing the cartridge and stably coupling and fixing the cartridge can allow the skin care device to be used semi-permanently.

In addition, the skin care device can sterilize the head that comes into contact with the skin, thereby maintaining a clean state.

Further, the skin care device can prevent the evaporation of the medium fluid within the cartridge, thereby preventing shortening of the lifespan of the cartridge.

The additional scope of applicability of the present disclosure will become apparent from the detailed description below. However, various changes and modifications within the spirit and scope of the present disclosure will be apparent to those skilled in the art. Thus, specific embodiments including the detailed descriptions and preferred embodiments of the present disclosure are given by way of example only.

### BRIEF DESCRIPTION OF THE DRAWINGS

The accompanying drawings, which are included to provide a further understanding of the disclosure and are incorporated in and constitute a part of this application, illustrate embodiment(s) of the disclosure and together with the description serve to explain the principle of the disclosure. In the drawings:

FIG. 1 is a perspective view showing a skin care device according to one embodiment of the present disclosure;

FIG. 2 is a perspective view showing a cartridge of the skin care device and a header of the skin care device when the cartridge is separated according to an embodiment of the present disclosure;

FIG. 3 is a view showing a coupling structure of the header of the skin care device according to an embodiment of the present disclosure;

FIG. 4 is a cross-sectional view of A-A and B-B of FIG. 1;

FIG. 5 is a cross-sectional view of the cartridge of the skin care device according to an embodiment of the present disclosure;

FIG. 6 is a view showing a head waterproofing portion of the skin care device according to an embodiment of the present disclosure;

FIG. 7 is a view showing a button waterproofing unit of the skin care device according to an embodiment of the present disclosure;

FIG. 8 is a perspective view showing the skin care device and a cradle according to an embodiment of the present disclosure;

FIG. 9 is a cross-sectional view of the header when the skin care device is seated on the cradle according to an embodiment of the present disclosure;

FIG. 10 is an enlarged view of the header and the head seating portion of the cradle of FIG. 9; and

FIG. 11 is a table showing the reduction of a medium fluid when a film is exposed to the outside and when the film is placed on the cradle having a double damper structure.

### DETAILED DESCRIPTION OF THE EMBODIMENTS

Description will now be given in detail according to embodiments disclosed herein, with reference to the accompanying drawings. For the sake of brief description with reference to the drawings, the same or equivalent components can be provided with the same reference numbers, and description thereof will not be repeated. In general, a suffix such as “module” and “unit” can be used to refer to elements or components. Use of such a suffix herein is merely intended to facilitate description of the specification, and the suffix itself is not intended to give any special meaning or function. In the present disclosure, that which is well-known to one of ordinary skill in the relevant art has generally been omitted for the sake of brevity. The accompanying drawings are used to help easily understand various technical features and it should be understood that the embodiments presented herein are not limited by the accompanying drawings. As such, the present disclosure should be construed to extend to any alterations, equivalents, and substitutes in addition to those which are particularly set out in the accompanying drawings.

It will be understood that although the terms first, second, etc. can be used herein to describe various elements, these elements should not be limited by these terms. These terms are generally only used to distinguish one element from another.

It will be understood that when an element is referred to as being “connected with” another element, the element can be directly connected with the other element or intervening elements can also be present. In contrast, when an element is referred to as being “directly connected with” another element, there are no intervening elements present.

A singular representation can include a plural representation unless it represents a definitely different meaning from the context.

Terms such as “include,” comprise, “has,” etc. are used herein and should be understood that they are intended to indicate an existence of several components, functions, or steps, disclosed in the specification, and it is also understood that greater or fewer components, functions, or steps can likewise be utilized.

Hereinafter, embodiments of the present disclosure will be described in detail with reference to the accompanying drawings.

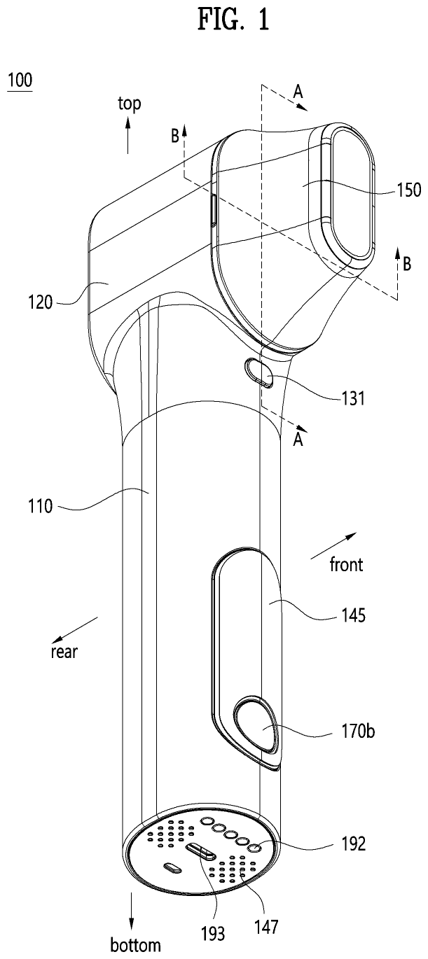

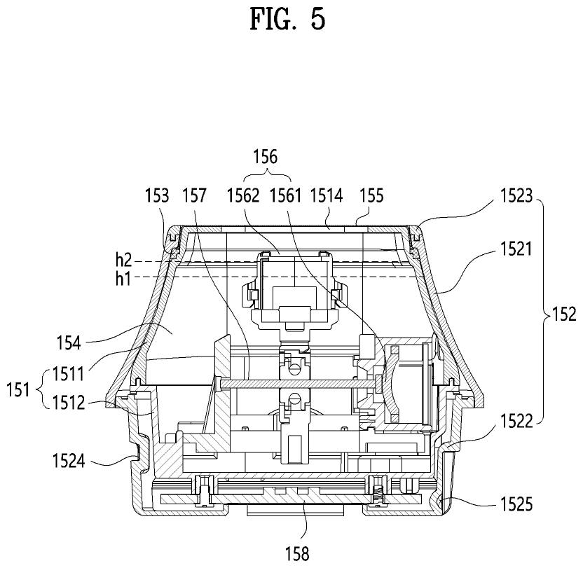

FIG. 1 is a perspective view showing a skin care device 100 according to one embodiment of the present disclosure. The skin care device 100 of the present disclosure includes a focused ultrasound device 156 (shown in FIG. 5) to promote collagen regeneration in the skin by supplying focused ultrasound energy to the skin.

The skin care device 100 can include a header 120 including an emitting surface 1513 for supplying ultrasound in close contact with the user's face. The skin care device 100 can include a handheld piece 110 to be held by the user's hand, such that the emitting surface 1513 is allowed to be in close contact with the user's skin.

Regarding the main body of the skin care device 100, a part where the emitting surface 1513 is located is referred to as the header 120, and a part having a cylindrical shape for easy gripping by the user is referred to as the handheld piece 110.

For convenience of explanation, the direction in which the emitting surface 1513 of the main body is located is referred to as the front, and the opposite direction is referred to as the rear. The portion in which the header 120 is located is called the top, and the opposite end of the handheld piece 110 is called the bottom.

A battery 190 can be mounted on the handheld piece 110, and power terminals 192 and 193 for connecting to an external power source can be provided to charge the battery 190. As shown in FIG. 1, the power terminals 192 and 193 are located at the bottom of the handheld piece 110, and the power terminals 192 and 193 can include terminal holes 193 to directly connect power cables. In addition, a charging terminal 192 for charging the battery 190 in contact with a power supply terminal 232, which is located on a cradle for holding the main body, can be provided.

The skin care device 100 can include an information display unit 145 (e.g., display) for providing information to the user. The information display unit 145 can display information, such as the number of times used and the number of remaining available times to the user. In addition, through the information display unit 145, the user can adjust settings, such as the intensity of ultrasound and the number of shots.

If there are predetermined modes configured by the manufacturer, those modes can be provided through the information display unit 145. Although it is shown that the information display unit 145 is located in the handheld piece, the present disclosure is not limited thereto. For example, the information display unit 145 can also be positioned on the rear of the header 120.

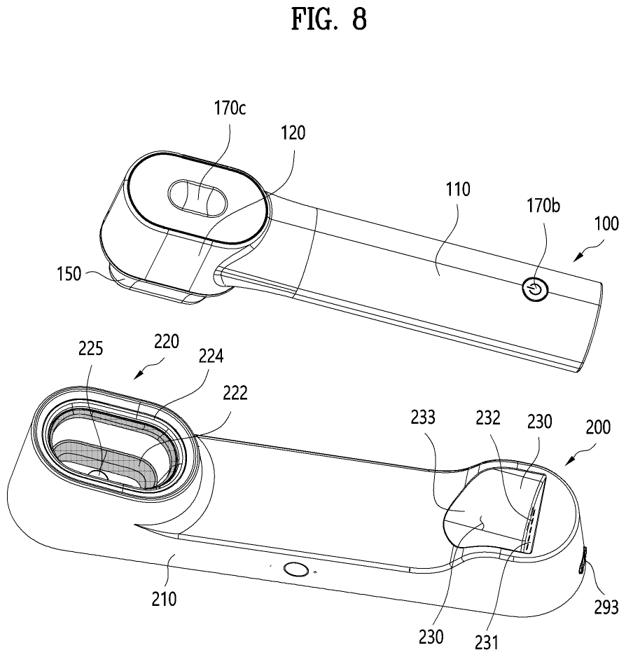

The skin care device 100 can use a touch sensor to receive user inputs. However, a physical button 170a (see FIG. 8) can be used for the power ON/OFF function because the skin care device 100 needs to be turned on while the power is turned off.

Instead of using a touch key, a button unit 170 that operates by physical force can be used such that the user can press the button unit 170 to drive the focused ultrasound device 156 without directly looking at the skin care device 100. A button unit 170c (see FIG. 8) for driving the focused ultrasound device 156 can be located on the rear of the header 120. In addition, a physical button 170b can be provided to adjust the level while in use.

The button unit 170 can include a switch 172 that generates an electrical signal by being pressed by physical force, such as a tactile switch 172 inside a case 111. To press the tactile switch 172, a button hole 117 can be formed in the main body case 111, and a retractable button top 173 can be inserted into the button hole 117. Alternatively, a portion of the case can be configured to have a material and thickness that is deformable when pressure is applied.

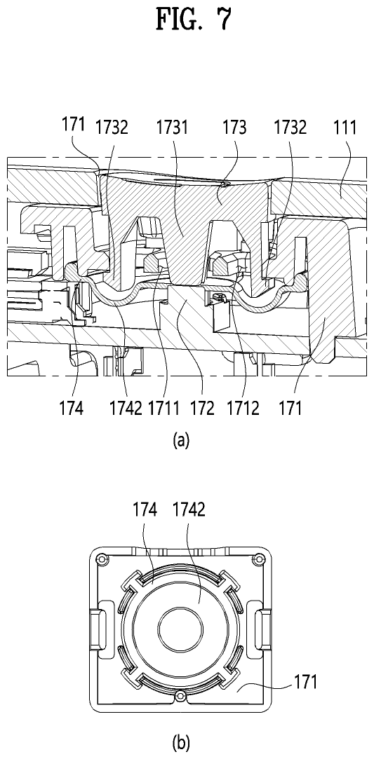

Details of the button unit 170 will be described later with reference to FIG. 7.

The focused ultrasound device 156 has a limited lifespan. Thus, the focused ultrasound device 156 can be degraded and become unusable after a certain number of uses. To address this issue, the focused ultrasound device 156 can be implemented in the form of a replaceable cartridge 150 capable of being replaced when its lifespan is exhausted.

The header 120 can be provided with a cartridge mounting portion 130 from which the ultrasonic cartridge 150 is detachable. The ultrasound cartridge 150 can be fastened to the cartridge mounting portion 130 and form a part of the header 120.

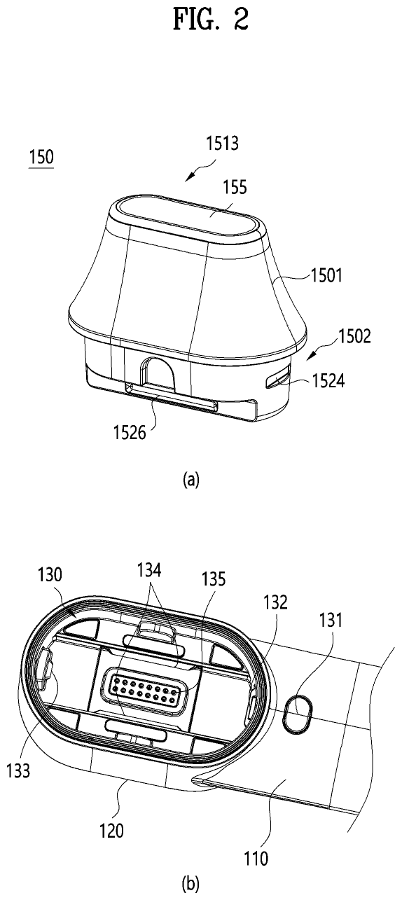

Here, (a) and (b) of FIG. 2 are perspective views showing the ultrasound cartridge 150 of the skin care device 100 and the cartridge mounting portion 130 of the skin care device 100 when the cartridge 150 is separated according to an embodiment of the present disclosure. Specifically, (a) of FIG. 2 shows the ultrasound cartridge 150, and (b) of FIG. 2 shows the cartridge mounting portion 130.

The ultrasound cartridge 150 can have an inclined surface 1501 gradually widening from the emitting surface 1513 to secure a mounting space for the focused ultrasound device 156 therein.

The ultrasound cartridge 150 can include a coupling portion 1502 inserted into the cartridge mounting portion 130. The coupling portion 1502 inserted into the cartridge mounting portion 130 can form a step with the inclined surface 1501 and have a shape corresponding to the shape of the cartridge mounting portion 130 such that inclined surface 1501 of the ultrasound cartridge 150 and the header 120 of the main body have an integral appearance.

In this embodiment, the focused ultrasound device 156 can have the long emitting surface 1513 that extends to one side as shown in FIG. 2 to provide focused ultrasound to the user's skin while moving in a straight direction. The focused ultrasound device 156 can also move in a two-dimensional plane and thus cover a wider area. In this case, the emitting surface 1513 can have a square or circular shape.

Referring to (b) of FIG. 2, the cartridge mounting portion 130 can include a cartridge hook 132 and elastic fastening protrusions 133 and 134, which are coupled to fastening grooves 1524, 1525, and 1526 formed in the ultrasound cartridge 150 such that the ultrasound cartridge 150 is fixed to the cartridge mounting portion 130.

To stably fix the ultrasound cartridge 150 and the cartridge mounting portion 130, the cartridge hook 132 and the elastic fastening protrusions 133 and 134 can be disposed in four directions. Since it is difficult to separate the ultrasound cartridge 150 and the cartridge mounting portion 130 if both are coupled with elastic fastening protrusions, the cartridge hook 132 capable of being drawn in and out by the user can be provided on one side.

Since it is easy to define a cartridge separation button 131 in a lower portion where the handheld piece 110 is located, the cartridge hook 132 can be located in a lower portion of the cartridge mounting portion 130, and the elastic fastening protrusion can be located in an upper portion and left and right portions.

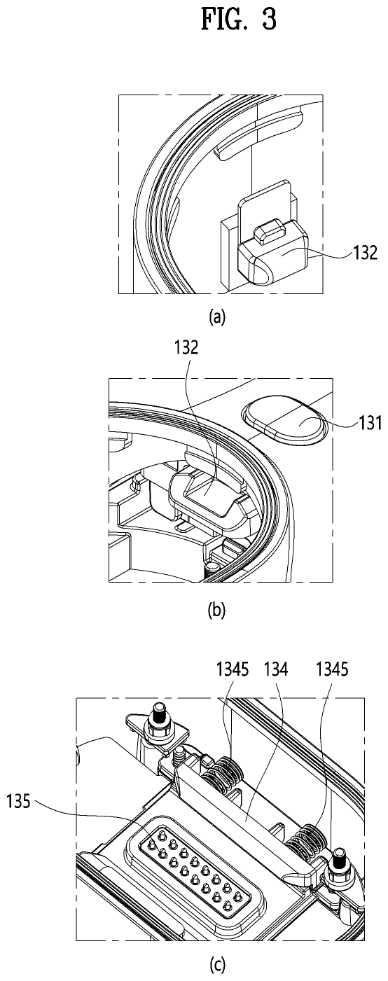

Here, (a)-(c) of FIG. 3 are views showing a coupling structure of the header 120 of the skin care device 100 according to an embodiment of the present disclosure. Specifically, FIG. 3 shows the cartridge hook 132 and the elastic fastening protrusions 133 and 134 when the inner cover of a head seating portion 220 is removed. (a) of FIG. 3 shows the first elastic fastening protrusion 133, (b) of FIG. 3 shows the cartridge hook 132, and (c) of FIG. 3 shows the second elastic fastening protrusion 134.

The first elastic fastening protrusion 133 facing the cartridge hook 132 has a small width due to constraints of the installation space and is fixed by the cartridge hook 132 in the vertical direction. Thus, the range of motion of the first elastic fastening protrusion 133 by elasticity can be small. Therefore, the first elastic fastening protrusion 133 can be implemented using the elasticity of a leaf spring to minimize the space as shown in FIG. 3.

The second elastic fastening protrusion 134 is symmetrically configured on both sides of the cartridge mounting portion 130. For stable fastening, the second elastic fastening protrusion 134 can have a longer shape than the first elastic fastening protrusion 133.

In this case, if a pair of springs 1345 arranged in the width direction are used as shown in (c) of FIG. 3 instead of using one elastic part, movement in the width direction can change. If force is provided on one side of the second elastic fastening protrusion 134 in the width direction, the one side can be drawn in further than the other side in the width direction of the second elastic fastening protrusion 134.

Each of the first elastic fastening protrusion 133 and second elastic fastening protrusion 134 can have a curved surface, and thus force can be transferred and introduced along the curved surface of the first elastic fastening protrusion 133, regardless of the direction of the force. In addition, the coupling force of the ultrasound cartridge 150 can be reduced when worn by friction. Thus, the first elastic fastening protrusion 133 and second elastic fastening protrusion 134 can be made of a polyoxymethylene (POM) material, which is resistant to abrasion.

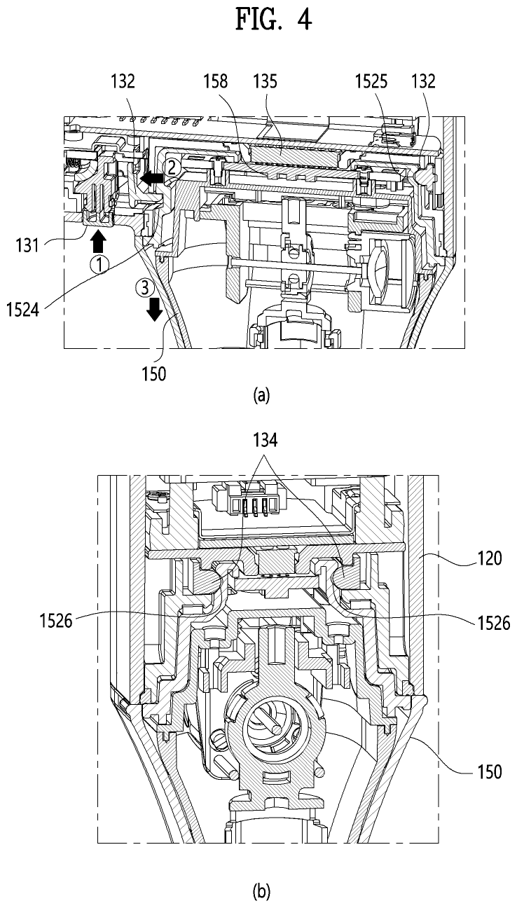

Here, (a) and (b) of FIG. 4 are cross-sectional views of A-A and B-B of FIG. 1, respectively.

When the cartridge separation button 131 located in a lower portion of the header 120 is pressed (\textcircled{1}), the cartridge hook 132 can be inserted (\textcircled{2}). Since the cartridge hook 132, which is a member for fixing one side of the ultrasound cartridge 150, is removed from the fastening groove 1524 of the ultrasound cartridge 150, a lower part (left side in the drawing) of the ultrasound cartridge 150 can be pulled out of the cartridge mounting portion 130.

Since the second elastic fastening protrusion 134 extends in the vertical direction, a lower portion of the second elastic fastening protrusion 134 can be inserted first and then an upper portion thereof can also be inserted when a lower portion of the ultrasound cartridge 150 is pulled out, as shown in (b) of FIG. 4. As a result, the ultrasound cartridge 150 can be separated from the cartridge mounting portion 130.

As shown in (a) of FIG. 4, the first elastic fastening protrusion 133 and the cartridge hook 132 can have different positions in the depth direction in the cartridge mounting portion 130.

The first elastic fastening protrusion 133 can serve to pressurize the ultrasound cartridge 150 such that the ultrasound cartridge 150 is fixed vertically in the cartridge mounting portion 130 with no gap. In addition, the first elastic fastening protrusion 133 can be positioned toward the inside of the cartridge mounting portion 130 to facilitate separation of the ultrasound cartridge 150.

The cartridge hook 132 can be positioned toward the outside of the cartridge mounting portion 130 to effectively limit the force for separating the ultrasound cartridge 150 from the cartridge mounting portion 130.

A cartridge connection terminal 158 for electrically connecting the ultrasound cartridge 150 and the body can be located on the opposite side of the emitting surface 1513, and a body connection terminal 135 can be located in a central portion surrounded by the elastic fastening protrusions and the cartridge hook 132. The emitting surface 1513 and the cartridge connection terminal 158 can be located on parallel surfaces to prevent connection failure while the skin care device 100 operates.

FIG. 5 is a cross-sectional view of the ultrasound cartridge 150 of the skin care device 100 according to an embodiment of the present disclosure. Specifically, FIG. 5 shows an internal configuration of the ultrasound cartridge 150.

The rear surface (bottom in FIG. 5) of the ultrasound cartridge 150 on which the cartridge connection terminal 158 is formed can be perpendicular to the emission direction of ultrasound (vertical direction in FIG. 5). This arrangement has an advantage in that there is no misalignment between the cartridge connection terminal 158 and the main body connection terminal 135 when focused ultrasound is injected. The ultrasound cartridge 150 includes a cartridge body 151 having an emitting hole 1514 defined on the emitting surface 1513. The focused ultrasound device 156 and a medium fluid 154 can be accommodated in the cartridge body 151.

The cartridge body 151 can be configured by combining two housings 1511 and 1512 for convenience of assembly and injection. In FIG. 5, a housing including the emitting surface 1513 is called a first cartridge housing 1511, and a part where the cartridge connection terminal 158 is located is called a second cartridge housing 1512. The focused ultrasound device 156 can be mounted on the second cartridge housing 1512. Then, the medium fluid 154 can be injected through the emitting hole 1514 after the first cartridge housing 1511 is coupled thereto.

The emitting hole 1514 can be blocked by a cover film 155 to cover the emitting hole 1514 such that the medium fluid 154 does not flow out. The cover film 155 can be made of a material that does not allow the medium fluid 154 to pass through but allows high-density focused ultrasound to pass through.

For example, the cover film 155 can be made of polyimide, but is not limited thereto. Polyimide is widely used as an insulating material having excellent mechanical strength and low permittivity.

The cover film 155 needs to completely seal the emitting hole 1514. Thus, the cover film 155 can be attached to the emitting surface 1513 by applying an adhesive to the emitting surface 1513 along the circumference of the emitting hole 1514. The gap between the cover film 155 and the cartridge body 151, which is located around the emitting surface 1513, as well as the gap between the cover film 155 and the emitting surface 1513 need to be eliminated as much as possible.

The emitting surface 1513 can be bonded to have a uniform thickness by pressing flat polyimide film materials, and the side portion thereof can be bonded to have a smooth surface by minimizing wrinkles as much as possible.

Since the first cartridge housing 1511 defines the exterior of the skin care device 100, a first cartridge cover 1521 for covering the first cartridge housing 1511 can be included. Apart in contact with the user's skin, that is, the circumference of the emitting surface 1513 can be made of a separate material such as metal.

Since the medium fluid 154 is located inside the cartridge body 151, the cartridge connection terminal 158, which needs to avoid contact with water, can be located outside the second cartridge housing 1512. To cover the cartridge connection terminal 158 located outside the second cartridge housing 1512, a second cartridge cover 1522 coupled to the outside of the second cartridge housing 1512 can be included.

The focused ultrasound device 156 can include a piezoelectric driver 1561 that generate high-intensity focused ultrasound and a piezoelectric ceramic 1562 disposed adjacent to the emitting hole 1514 to emit high-intensity focused ultrasound into the emitting hole 1514.

The piezoelectric ceramic 1562 can include a movable ceramic portion 1563 to provide high-intensity focused ultrasound to a predetermined area when the emitting surface 1513 is in contact with the user's skin.

As a medium that transfers high-density focused ultrasound emitted from the piezoelectric ceramic 1562 to the user's skin, the medium fluid 154 can be accommodated within the cartridge body 151. Water needs to be placed between the piezoelectric ceramic 1562 and the emitting hole 1514 to transfer high-intensity focused ultrasound to the user's skin.

Water can be used as the medium fluid 154. However, when water freezes at temperatures below zero and becomes a solid, the volume can increase. When the medium fluid 154 is at a temperature below zero while the medium fluid 154 is in contact with the piezoelectric ceramic 1562, the medium fluid 154 freezes and increases in volume, which can cause damage to the piezoelectric ceramic 1562. When the user uses the skin care device 100, the user can use the skin care device 100 indoors, and thus there is no risk of exposure to temperatures below zero. However, the skin care device 100 can be placed in sub-zero temperatures during transport.

To prevent damage to the piezoelectric ceramic 1562, if the medium fluid 154 is filled up to a height of h1 (about 86 to 90% of the internal space of the ultrasound cartridge 150) as shown in FIG. 5, the volume of the medium fluid 154 can increase only up to h2 even when frozen, thereby protecting the piezoelectric ceramic 1562.

Therefore, the skin care device 100 can be packaged such that the emitting surface 1513 of the cartridge 150 faces upwards. In addition, it can be noticed that the packaging of the skin care device 100 needs to be maintained in the same orientation as the box is packaged without tilting or turning over.

When the user uses the skin care device 100, the medium fluid 154 needs to be positioned between the piezoelectric ceramic 1562 and the emitting hole 1514 to allow high-density focused ultrasound to be transferred to the user's skin. However, as described above, when the emitting surface 1513 of the skin care device 100 is in parallel with the ground, the medium fluid 154 may not exist between the piezoelectric ceramic 1562 and the emitting hole 1514 because the cartridge body 151 is not completely filled with the medium fluid 154.

The skin care device 100 can use a sensor to detect whether the medium fluid 154 is positioned between the piezoelectric ceramic 1562 and the emitting hole 1514. The skin care device 100 can stop driving the focused ultrasound device 156 when the medium fluid 154 is not positioned between the piezoelectric ceramic 1562 and the emitting hole 1514.

Alternatively, the skin care device 100 can have a speaker in the main body to notify the user of a posture corresponding to an invalid shot. Referring to FIG. 1, a hole 147 (e.g., or a plurality of holes 147) for the speaker can be defined at the bottom of the main body. A simple warning sound can be used, but the skin care device 100 can use a voice guidance system to provide specific instructions to change the posture.

A sensor can be provided in the emitting hole 1514 to detect the presence or absence of the medium fluid 154. When a gyroscope (gyro) sensor is provided on the main body, if the main body is tilted at an angle at which the medium fluid 154 does not exist between the emitting hole 1514 and the piezoelectric ceramic 1562, it can be determined as the posture (e.g., orientation) corresponding to the invalid shot.

A controller can determine whether to drive the focused ultrasound device 156 based on body posture information (e.g., orientation) detected by the gyro sensor. The controller can guide the user to adjust the inclination of the main body.

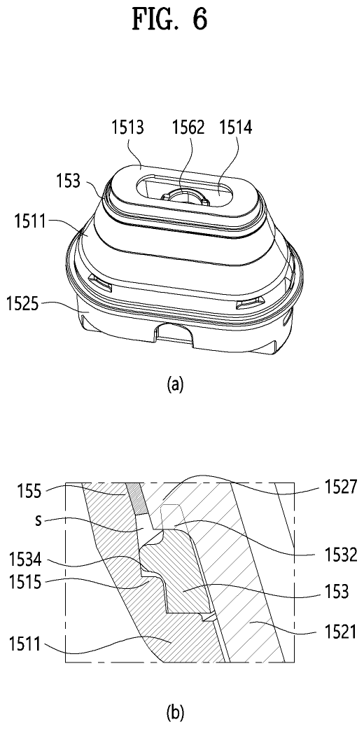

Here, (a) and (b) of FIG. 6 are views showing ahead waterproofing portion of the skin care device 100 according to an embodiment of the present disclosure. Specifically, (a) of FIG. 6 shows a state in which the cover film 155 is separated from the first cartridge cover 1521 of the ultrasound cartridge 150 shown in FIG. 2.

The emitting hole 1514 can have a long elliptical shape extending to one side as shown in (a) of FIG. 6. The emitting hole 1514 can include the cover film 155 (see FIG. 5) covering the emitting hole 1514 to prevent the medium fluid 154 injected therein from flowing out.

The skin care device 100 can emit high-density focused ultrasound after liquid cosmetics, such as moisture gel, are applied to the skin. The inside of the cartridge body 151 is covered by the cover film 155 made of a waterproof material, and thus liquid cosmetics do not flow in. The skin care device 100 can break down if water enters electronic components located outside the cartridge body 151 such as the connection terminals.

Therefore, a waterproof ring 153 can be disposed on the side of a cartridge cover 152 adjacent to the emitting surface 1513 to prevent liquid from flowing between the cartridge cover 152 and the cartridge body 151.

The waterproof ring 153 can be interposed between the first cartridge housing 1511 and the first cartridge cover 1521 to prevent liquid from flowing into a gap next to the emitting surface 1513. As shown in (b) of FIG. 6, the first cartridge cover 1521 can include an L-shaped first step portion 1527 therein. When the waterproof ring 153 is compressed in the first step portion 1527, it can increase the contact area between the waterproof ring 153 and the first cartridge cover 1521.

In particular, the waterproof ring 153 can include a protrusion 1532 that protrudes and overlaps with the first step portion 1527. When the first cartridge cover 1521 is coupled to the cartridge body 151, the protrusion 1532 can be compressed and fill an empty space s between the first cartridge housing 1511 and the first cartridge cover 1521, thereby increasing the waterproof effect.

In this embodiment, since the first cartridge housing 1511 of the cartridge body 151 gradually becomes wider toward the rear from the emitting surface 1513, it can be difficult to insert the waterproof ring 153 if it has a U-shaped cross-section.

Therefore, as shown in (b) of FIG. 6, an L-shaped second step portion 1515 can also be formed in the cartridge body 151, and the lower surface of the waterproof ring 153 can be configured to have an L-shape 1534 corresponding to that of the second stepped portion 1515.

The waterproof ring 153 interposed between the first step portion 1527 of the cartridge cover 152 and the second step portion 1515 of the cartridge body 151 can have a wide contact area with the two members. In addition, the waterproof ring 153 can also have an excellent waterproof effect because the protrusion 1532 is compressed and adhered.

(a) and (b) of FIG. 7 are views showing the button unit 170 of the skin care device 100 according to an embodiment of the present disclosure. Specifically, (a) of FIG. 7 shows a cross-section of the button unit 170. The button unit 170 can be implemented with a tactile or dome switch 172 to enhance the user experience.

The button unit 170 requires physical movement. Accordingly, the button hole 117 can be formed in the housing of the main body, and the button unit 170 can include: the button top 173 that is inserted into the hole, and the switch 172 that is compressed when pressure is applied to the button top 173. Since the button top 173 needs to be drawn in and out of the button hole 117 formed in the body housing 111 without any foreign matter, there is a gap between the button top 173 and the button hole 117 in the body housing 111.

To prevent foreign substances from entering through the gap, it is necessary to have a certain level of waterproofing functionality. For example, the foreign substances can potentially cause malfunctions in electronic components such as the battery 190 or a circuit board located inside the main body. Since the perimeter of the emitting surface 1513 of the header 120 comes into direct contact with liquid cosmetics, tight waterproofing is required. However, the button unit 170 requires a waterproofing level lower than that of the perimeter of the emitting surface 1513. In addition to waterproofing functionality, it is important to consider the smooth operation of the button unit 170 to ensure that the movement of the button top 173 is not hindered.

Further, (b) of FIG. 7 is a view showing a button bracket 171 located between the button top 173 and the switch 172.

The button bracket 171 can include: a hook hole 1711 into which a button hook 1732 of the button top 173 is inserted to fix the button top 173 to the body housing 111, and a switch hole 1711 through which a tactile boss 1731 penetrates to be in contact with the switch 172.

As shown in (a) of FIG. 7, the button bracket 171 can include a hook hole 1712 in which the button hook 1732 that protrudes from the button top 173 is caught. The button hook 1732 is caught in the hook hole 1712, and thus the button top 173 can be securely fastened to prevent the button top 173 from detaching from the button hole 117.

The button bracket 171 can include an elastic cover 174 to prevent liquid from flowing through the button hole 117 while fixing the button top 173. The elastic cover 174 is coupled to the button bracket 171. When the button bracket 171 is assembled, the elastic cover 174 covers a lower portion exposed by the button hole 117 as shown in (a) of FIG. 7.

To prevent interference between the button hook 1732 and the elastic cover 174, which is caused when pressure is applied to the button top 173, the elastic cover 174 can include a groove 1742 drawn inward (e.g., concave inward) at a position corresponding to the button hook 1732. In this embodiment, a donut-shaped groove 1742 is included as shown in (b) of FIG. 7.

The elastic cover 174 can be made of an elastic material, such as silicone, and the elastic cover 174 can be interposed between the switch 172 and the tactile boss 1731 for pressing the switch 172 on the button top 173.

Since the tactile switch 172 is elastic, the button top 173 can protrude when the force pressing the button top 173 is removed. In addition, the elasticity of the elastic cover 174 can also provide force to restore the button top 173 to its original position.

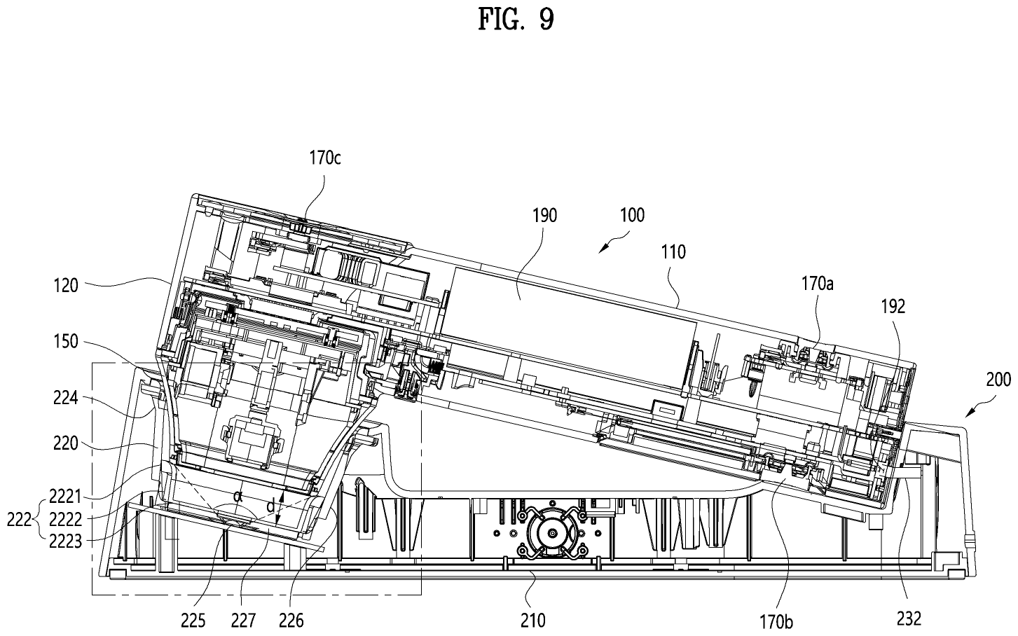

FIG. 8 is a perspective view showing the skin care device 100 and a cradle 200 according to an embodiment of the present disclosure. FIG. 9 is a cross-sectional view of the skin care device 100 and the cradle 200 in a state in which the skin care device 100 is seated on the cradle 200 according to an embodiment of the present disclosure. The skin care device 100 according to the present disclosure can include the cradle 200 that holds the main body.

The cradle 200 can be configured in an upright position. However, since the weight of the header 120 is relatively heavier than that of the handheld piece 110, the skin care device 100 can tip over. Therefore, according to the present disclosure, the cradle 200 can have a form that supports both the upper and lower parts of the main body simultaneously.

The cradle 200 can include: a base portion 210 that is mounted on the floor, the head seating portion 220 positioned on one side of the base portion 210, and a lower support portion 230 positioned on the other side. The head seating portion 220 is a part where the header 120 of the main body is attached. The header 120 of the main body is inserted such that the emitting surface 1513 faces a bottom surface 227 of the head seating portion 220. Additionally, at least a portion of the ultrasound cartridge 150 can be stored within the head seating portion 220.

The lower support portion 230 includes the power supply terminal 232. The bottom of the handheld piece 110 can be inserted into the lower support portion 230 such that the power supply terminal 232 aligns with the charging terminal 192 located at the bottom of the handheld piece 110.

The head seating portion 220 can include: the seating portion bottom surface 227 that faces the emitting hole 1514 of the header 120, and a seating portion side surface 226 extended and connected to the perimeter of the seating portion bottom surface 227. Since the cartridge 150 of the header 120 includes the inclined surface 1501 that widens from the emitting surface 1513, the seating portion side surface 226 can also have an inclined surface corresponding to the shape of the header 120.

In addition to simply supporting the header 120 of the main body, the head seating portion 220 can also serve the following functions: preventing the evaporation of the medium fluid 154 through the emitting hole 1514 by covering the emitting hole 1514 when the main body is placed on the cradle 200, and sterilizing the emitting surface 1513.

The cover film 155 that covers the emitting hole 1514 has the waterproof capability, but the medium fluid 154, which is water, can evaporate when the skin care device 100 is placed for a long time. When the ultrasound cartridge 150 is left exposed to the outside, approximately 0.03 g of medium fluid 154 can evaporate per day. As a result, the performance of the skin care device 100 can be degraded due to lack of the medium fluid 154.

Accordingly, according to the present disclosure, when the skin care device 100 is placed on the cradle 200, the emitting hole 1514 can be shielded from the outside to prevent water from evaporating through the emitting hole 1514. To prevent water from evaporating through the emitting hole 1514, the sidewall of the head seating portion 220 can include damper rings 222 and 224 that are in close contact with the sidewall of the ultrasound cartridge 150 attached to the head seating portion 220.

As shown in FIG. 8, the damper rings 222 and 224 can be made of an elastic member and have a ring shape along the seating portion side surface 226 of the head seating portion 220. In addition, dual damper rings 222 and 224 can be included to increase the sealing effect.

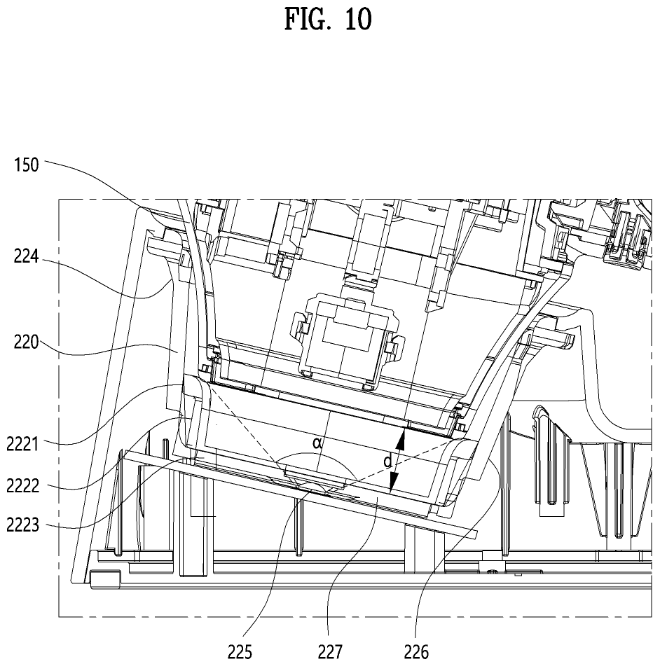

FIG. 10 is an enlarged view of the header 120 and the head seating portion 220 of the cradle 200 of FIG. 9.

Referring to FIG. 10, the first damper ring 222 located close to the seating portion bottom surface 227 can come into contact with the circumference of the emitting surface 1513 of the cartridge 150. Therefore, an upper portion 2221 of the first damper ring 222 can have a thick width such that the circumference of the emitting surface 1513 is seated thereon.

The first damper ring 222 can be extended to a connecting portion between the seating portion side surface 226 and the seating portion bottom surface 227 to prevent the evaporation of medium fluid 154 through the connecting portion. For example, a lower portion 2223 of the first damper ring 222 can be interposed at the connecting portion between the seating portion side surface 226 and the seating portion bottom surface 227.

The emitting surface 1513, which is located above the first damper ring 222, can be positioned at a certain distance from the seating portion bottom surface 227. The first damper ring 222 can include a middle portion 2222 that connects the lower and upper portions of the first damper ring 222 to define the height of the first damper ring 222 corresponding to the distance from the seating portion bottom surface 227 and extends along the inside of the seating portion side surface 226. The radial width of the middle portion 2222 can be narrower than the radial width of the upper portion 2221.

Since the first damper ring 222 is in contact with the emitting surface 1513, the second damper ring 224 in contact with the side of the cartridge 150 can be further included to provide better protection against the evaporation of the medium fluid 154.

The second damper ring 224 can be positioned in the direction of the entrance of the head seating portion 220, relative to the first damper ring 222. The second damper ring 224 can have a curved surface protruding inwards to ensure contact even if the angle of the header 120 is slightly misaligned.

When the dual damper rings 222 and 224 are applied, the evaporation rate of the medium fluid 154 can be effectively reduced.

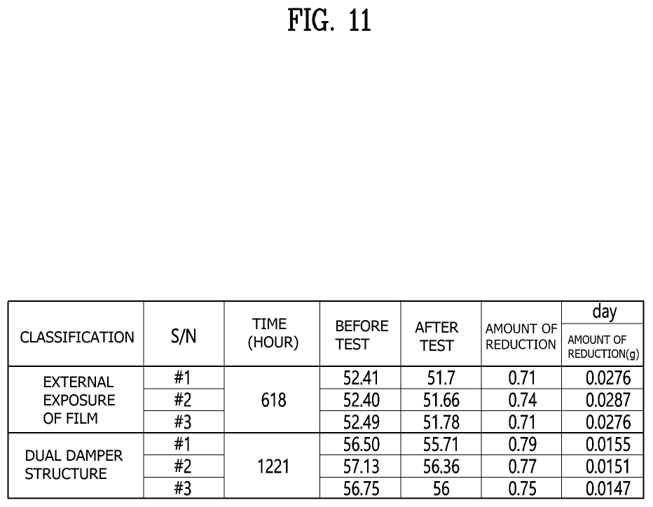

FIG. 11 is a table showing the reduction of the medium fluid 154 when a film is exposed to the outside and when the film is placed on the cradle 200 having a double damper structure.

When the skin care device 100 is placed on the cradle 200, the amount of reduction can become similar even if it is left for approximately twice the time. Accordingly, the amount of evaporation per day can be reduced by half to 0.015 g, thereby preventing shortening of the lifespan of the cartridge 150 due to the evaporation of the medium fluid 154.

When the header 120 is inserted into the upper portion 2221 of the first damper ring 222, that is, the head seating portion 220, the emitting surface 1513 and the bottom surface 227 of the head seating portion 220 are positioned at a first distance apart. This arrangement is to allow the UV light from a sterilization lamp 225, located on the seating portion bottom surface 227, to be irradiated to the entirety of the emitting surface 1513. The first distance d can be determined in consideration of the irradiation angle of the sterilization lamp 225 to ensure that the UV light is irradiated to the entirety of the emitting surface 1513.

If the size of the irradiation diameter of the sterilization lamp 225 is larger than the inner circumference of the upper portion 2221 of the first damper ring 222 at the first distance, which depends on the irradiation angle of the sterilization lamp 225, the emitting surface 1513 can be sufficiently sterilized.

The middle portion 2222 of the first damper ring 222 can include a tapered surface that narrows towards the lower portion 2223. To ensure that there is no interference with the UV light, the angle of the tapered surface in the middle portion 2222 of the first damper ring 222 can be smaller than the irradiation angle of the sterilization lamp 225.

The bottom surface of the header 120 can be parallel to the bottom surface of the base portion 210. However, in that case, if the skin care device 100 is placed on the cradle 200, the overall height increases. Therefore, as shown in FIG. 9, the skin care device 100 can be placed on the cradle 200 at an angle such that the header 120 of the skin care device 100 is tilted slightly upwards. As a result, the emitting surface 1513 also tilts in accordance with the inclination of the handheld piece 110, which allows the seating portion bottom surface 227 to be inclined at a certain angle.

Since the handheld piece 110 is inclined, the lower support portion 230 can also have an inclined seating surface corresponding to the tilt of the handheld piece 110. A first seating surface 231, where the power supply terminal 232 connected to the charging terminal 192 located at the bottom of the handheld piece 110 is located, can be perpendicular to the seating portion bottom surface 227.

Since the front lower portion of the handheld piece 110 is attached to the lower support portion 230 in the tilted state as shown in FIG. 9, a second seating surface 233 having a curved surface corresponding to the curved surface of the handheld piece 110 can be included.

According to an embodiment of the present disclosure, the structure for stably coupling and fixing the cartridge 150 to the skin care device 100 can allow the skin care device 100 to be used semi-permanently.

In addition, the skin care device 100 can sterilize the emitting surface 1513 of the header 120 that comes into contact with the skin, thereby maintaining a clean state.

Further, the skin care device 100 can prevent the evaporation of the medium fluid 154 within the cartridge 150, thereby preventing shortening of the lifespan of the cartridge 150.

The above description is merely an illustrative example of the technical concepts within the scope of the present disclosure, and it should be understood that various modifications and variations are possible within the ordinary knowledge of those skilled in the art to which the present disclosure pertains, without departing from the essential characteristics of the present disclosure.

Therefore, the embodiments disclosed in the present disclosure are not intended to limit the technical concepts of the present disclosure but are provided for illustrative purposes. It should be understood that the scope of the technical concepts of the present disclosure is not limited by the embodiments.

Various embodiments described herein may be implemented in a computer-readable medium using, for example, software, hardware, or some combination thereof. For example, the embodiments described herein may be implemented within one or more of Application Specific Integrated Circuits (ASICs), Digital Signal Processors (DSPs), Digital Signal Processing Devices (DSPDs), Programmable Logic Devices (PLDs), Field Programmable Gate Arrays (FPGAs), processors, controllers, micro-controllers, microprocessors, other electronic units designed to perform the functions described herein, or a selective combination thereof. In some cases, such embodiments are implemented by the controller. For Example, the controller is a hardware-embedded processor executing the appropriate algorithms (e.g., flowcharts) for performing the described functions and thus has sufficient structure. Also, the embodiments such as procedures and functions may be implemented together with separate software modules each of which performs at least one of functions and operations. The software codes can be implemented with a software application written in any suitable programming language. Also, the software codes can be stored in the memory and executed by the controller, thus making the controller a type of special purpose controller specifically configured to carry out the described functions and algorithms. Thus, the components shown in the drawings have sufficient structure to implement the appropriate algorithms for performing the described functions.

For a software implementation, the embodiments such as procedures and functions may be implemented together with separate software modules each of which performs at least one of functions and operations. The software codes can be implemented with a software application written in any suitable programming language. Also, the software codes may be stored in the memory and executed by the controller. Thus, the components shown in the drawings have sufficient structure to implement the appropriate algorithms for performing the described functions.

The scope of the present disclosure should be interpreted in accordance with the claims below, and all technical concepts within the scope equivalent thereto should be interpreted as falling within the scope of the rights of the present disclosure.

What is claimed is:

1. A cradle configured to hold a skin care device, wherein the skin care device includes a header configured to accommodate a medium fluid and the header has an emitting hole positioned on an emitting surface, the cradle comprising:
a head seating portion configured to receive the header; and
a damper ring disposed around a side perimeter of the head seating portion, the damper ring being configured to attach to a perimeter of the header,
wherein the head seating portion includes:
a seating portion bottom surface configured to face the emitting hole of the header; and
a seating portion side surface connected to a perimeter of the seating portion bottom surface, and
wherein the damper ring is configured to prevent evaporation of the medium fluid by covering a gap between the head seating portion and the header.

2. The cradle of claim 1, wherein the damper ring includes a first damper ring configured to contact a perimeter of a surface on which the emitting hole of the header is formed,
wherein the first damper ring includes an upper portion configured to contact the skin care device, and
wherein the upper portion is positioned a first distance away from the seating portion bottom surface.

3. The cradle of claim 2, wherein the first damper ring comprises:
a middle portion extending downward from the upper portion along an inside of the seating portion side surface; and
a lower portion interposed at a connecting portion between the seating portion bottom surface and the seating portion side surface.

4. The cradle of claim 2, further comprising a sterilization lamp positioned on the seating portion bottom surface and configured to irradiate ultraviolet (UV) light at a first irradiation angle, and
wherein a size of an irradiation diameter of the sterilization lamp at the first distance is greater than a size of an inner perimeter of the upper portion.

5. The cradle of claim 4, wherein the first damper ring includes a middle portion tapered downward from the upper portion at an inclination angle, and
wherein the inclination angle of the middle portion is smaller than the first irradiation angle of the sterilization lamp.

6. The cradle of claim 1, wherein the header further includes a cartridge side surface having a slope that narrows towards the emitting surface,
wherein the seating portion side surface narrows towards the seating portion bottom surface in accordance with the cartridge side surface, and
wherein the damper ring comprises a second damper ring in contact with the cartridge side surface.

7. The cradle of claim 6, wherein the second damper ring has a curved surface protruding inwards and evenly contacts the header.

8. The cradle of claim 1, further comprising:
a base portion, wherein the head seating portion is positioned on a first side of the base portion;
a lower support portion positioned on a second side of the base portion; and
a power supply terminal positioned in the lower support portion, the power supply terminal configured to contact a charging terminal of the skin care device.

9. The cradle of claim 8, wherein the seating portion bottom surface is not parallel to the base portion,
wherein the power supply terminal is positioned on a first seating surface, and
wherein the first seating surface is perpendicular to the seating portion bottom surface.

10. A skin care device, comprising:
a main body;
an ultrasound cartridge positioned on one side of the main body; and
a cradle including:
a head seating portion; and
a damper ring disposed around a perimeter of the head seating portion, the damper ring being positioned against a perimeter of the ultrasound cartridge,
wherein the ultrasound cartridge includes:
a cartridge body including:
an emitting hole formed on an emitting surface; and
an internal cavity connected to the emitting hole;
a cover film covering the emitting hole;
a piezoelectric driver mounted in the internal cavity, the piezoelectric drive being configured to generate high-intensity focused ultrasound;
a piezoelectric ceramic configured to emit the high-intensity focused ultrasound toward the emitting hole of the cartridge body; and
an ultrasonic medium fluid accommodated in the internal cavity.

11. The skin care device of claim 10, wherein the head seating portion comprises:
a seating portion bottom surface facing the emitting hole of the cartridge body; and
a seating portion side surface connected to a perimeter of the seating portion bottom surface, wherein the damper ring is disposed inside the seating portion side surface.

12. A skin care device, comprising:
a main body including a cartridge seating portion; and
an ultrasound cartridge detachable from the cartridge seating portion, the ultrasound cartridge including:
a cartridge body including:
an internal cavity; and
an emitting hole formed on an emitting surface;
a cover film covering the emitting hole;
a piezoelectric driver mounted in the internal cavity, the piezoelectric drive being configured to generate high-intensity focused ultrasound;
a piezoelectric ceramic configured to emit the high-intensity focused ultrasound toward the emitting hole of the cartridge body; and
an ultrasonic medium fluid accommodated in the internal cavity,
wherein the ultrasonic medium fluid is filled up to a position lower than a height of the piezoelectric ceramic, when the emitting hole is oriented upward.

13. The skin care device of claim 12, wherein the ultrasound cartridge further includes:
a waterproof ring fitted around a perimeter of the cartridge body, the waterproof ring including a protrusion having an outer perimeter protruding upwards; and
a cartridge cover inserted from a first side of the cartridge body on which the cover film is positioned,
wherein the cartridge cover includes a first step portion inside the cartridge cover, wherein a lower portion of the first step portion is drawn toward an outside thereof, and
wherein the protrusion of the waterproof ring is deformed by being pressed on a step surface of the first step portion to fill a space between the cartridge body and the cartridge cover.

14. The skin care device of claim 13, wherein the cartridge body further includes a second step portion formed on an outer perimeter of the cartridge body, and
wherein a cross section of the waterproof ring has an L-shaped lower side surface surrounding upper and side surfaces of the second step portion.

15. The skin care device of claim 13, wherein the cover film covers the emitting surface of the cartridge body,
wherein the cover film covers a side surface positioned around a perimeter of the emitting surface, and
wherein the first step portion is positioned below an end of the cover film.

16. The skin care device of claim 12, further comprising a release button,
wherein the cartridge seating portion includes:
a cartridge hook protruding from a first side, the cartridge hook being inserted inward based on a pressing of the release button; and
a first elastic fastening protrusion protruding from a second side opposite to the first side and being inserted based on pressurization.

17. The skin care device of claim 16, wherein the cartridge seating portion further includes a pair of second elastic fastening protrusions,
wherein the second elastic fastening protrusions are positioned between the first side and the second side,
wherein the second elastic fastening protrusions protrude symmetrically and are inserted based on pressurization, and
wherein each of the first elastic fastening protrusion and the second elastic fastening protrusion has a curved surface.

18. The skin care device of claim 17, wherein each second elastic fastening protrusion is longer than each first elastic fastening protrusion, and
wherein the pair of second elastic fastening protrusions include a pair of springs arranged in parallel along a length direction of the pair of second elastic fastening protrusions.

19. The skin care device of claim 12, further comprising:
a button hole formed on the main body;
a button bracket positioned inside the button hole, the button bracket including:
a switch hole; and
a hook hole; and
a button top positioned in the button hole, the button top including:
a button hook inserted and hooked into the hook hole; and
a tactile boss inserted into the switch hole.

20. The skin care device of claim 12, further comprising:
a gyroscope sensor provided on the main body, the gyroscope sensor being configured to measure an orientation of the main body; and
a controller configured to:
calculate a surface level of the ultrasonic medium fluid within the cartridge body based on the orientation of the main body; and
limit driving of the piezoelectric driver when there is no ultrasonic medium fluid between the piezoelectric ceramic and the emitting hole of the cartridge body.
